(19) United States
(12) Patent Application Publication
(10) Pub. No.: US 2009/0076367 A1
(43) Pub. Date: Mar. 19, 2009

(54) Monitoring Intraocular Pressure

(75) Inventors: Arthur J. Sit, Rochester, MN (US); Jay W. McLaren, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research, Rochester, MN (US)

(21) Appl. No.: 12/272,180

(22) Filed: Nov. 17, 2008

Related U.S. Application Data
(63) Continuation-in-part of application No. PCT/US2007/068536, filed on May 9, 2007.
(60) Provisional application No. 60/801,008, filed on May 17, 2006.

(51) Int. Cl. A61B 3/16

(52) U.S. Cl. 600/398

(57) ABSTRACT

Some embodiments of the system described herein provide non-invasive intraocular pressure monitoring throughout an extended period. Such monitoring systems can be used for the diagnosis and management of glaucoma patients and those at risk for glaucoma. In some embodiments, the monitoring system provides intraocular pressure monitoring in the patient's normal environment without the need to house the patient in a sleep laboratory.

### CROSS-REFERENCE TO RELATED APPLICATIONS

This application is a continuation-in-part application of and claims priority to International Application Serial No. PCT/US2007/068536, having an International Filing Date of May 9, 2007, which claims priority to U.S. Patent Application No. 60/801,008 filed on May 17, 2006 and entitled “Monitoring Intraocular Pressure,” the entire contents of which is incorporated herein by reference.

### TECHNICAL FIELD

This document relates to systems and methods for monitoring intraocular pressure.

### BACKGROUND

Intraocular pressure is a risk factor for the development and progression of glaucoma or other visual impairment conditions. Reduction of intraocular pressure has been shown to reduce the risk of developing glaucoma as well as the risk of disease progression. A portion of glaucoma patients continue to experience visual deterioration even with apparently well controlled intraocular pressure based on periodic visits (e.g., visits every three to six months) to a clinic to monitor and control their intraocular pressure.

In some circumstances, a patient with well-controlled intraocular pressure during clinic office hours may experience intraocular pressure peaks at other times in the day. Accordingly, it is believed that intraocular pressure fluctuations may account for some of the cases of progressive damage in patients with intraocular pressures that appear to be controlled during periodic visits to the clinic. Indeed, some investigators have suggested that fluctuations in intraocular pressure may be an independent risk factor for progression of disease.

In one example of intraocular pressure fluctuations that are not normally detected by daytime visits to a clinic, a patient's intraocular pressure may fluctuate and reach higher levels during the nocturnal period (e.g., during sleep) than in the diurnal period. Combined with a decrease in blood pressure that occurs during the nocturnal period, the increase in nocturnal intraocular pressure may compromise optic nerve head blood flow in susceptible individuals.

Because the periodic visits to a clinic may not detect the intraocular pressure fluctuations throughout the day, some patients have used sleep laboratories to monitor intraocular pressure over a 24-hour period. Such a monitoring technique can be logistically difficult for normal patient care.

### SUMMARY

Some embodiments of the system described herein provide non-invasive intraocular pressure monitoring throughout an extended period (e.g., a 6-hour period, a 12-hour period, a 24-hour period, or more). Such monitoring systems can be used for the diagnosis and management of glaucoma patients and those at risk for glaucoma. In some embodiments, the monitoring system provides intraocular pressure monitoring in the patient's normal environment without the need to house the patient in a sleep laboratory. In these circumstances, the management of glaucoma patients can be improved by allowing identification of patients with large fluctuations in intraocular pressure, or intraocular pressure elevations outside of the clinic office hours. Treatment may then be modified appropriately to prevent the deterioration in the condition of the patient's vision.

In some embodiments, the monitoring system described herein may include a contact lens device that is removably engageable with a user's eye. The contact lens device may include a sensor device to detect when a deformable portion of the contact lens device is indented by a predetermined amount. The monitoring system may also include a headset device that applies a force to indent the deformable portion of the contact lens device. The headset device may include at least one force sensor coupled to a headset frame. The monitoring system may further include a control system to activate the headset device to apply the force on the contact lens device. The control system may be in electrical communication with the force sensor to record data from the force sensor when the contact lens device is indented by the predetermined amount.

The force measurements can be recorded at a regular interval (e.g., every five minutes, every ten minutes, every twenty minutes, every 60 minutes, or the like) and transmitted to a computer system for subsequent calculations (e.g., intraocular pressure calculations or the like) and display (e.g., an intraocular pressure profile showing intraocular pressure measurements as a function of time). As such, glaucoma patient management may involve collecting an intraocular pressure profile recorded over at least a 24-hour period. Such an intra-ocular pressure profile can be used at the initial diagnosis stage, when changing a patient's therapy to assess efficacy, or annually to monitor the intraocular pressure control.

Some or all of the embodiments described herein may include one or more of the following advantages. First, the monitoring system can provide measurement of intraocular pressure throughout a 24-hour period without the need for the patient to be kept in a hospital or in a sleep laboratory (which can result in the loss of a full day of activities or work for the patient). Thus, a patient may be able to continue normal activities while the intraocular pressure monitoring system is operational. Second, the monitoring system can provide passive measurements (e.g., no required activation step by the patient), so the system is capable of monitoring the intraocular pressure even when the patient is asleep. Third, some embodiments of the system are capable of monitoring the intraocular pressure regardless of whether the patient's eyelids are opened or closed. Fourth, the monitoring system can be implemented without an invasive surgical procedure. Fifth, some embodiments of the system can be operated with minimal patient interaction and without the need for complex training of the patient. Sixth, the force sensor (or pressure sensor) may be disposed on the headset (rather than embedded in on the contact lens itself), which can reduce the complexity of the design and reduce manufacturing costs. Seventh, the monitoring system may be a robust design that can provide substantially accurate measurements regardless of normal eye movements, thereby reducing some error-causing effects from measurement noise. Eighth, the monitoring system may benefit the eye-care provider by freeing staff from the time-consuming practice of serial tonometry during patient visits to the eye clinic.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-B are partial cross-sectional views of a monitoring system, in accordance with some embodiments.

FIG. 2 is a front view of a contact lens device of the monitoring system of FIGS. 1A-B.

FIG. 3 is a cross-sectional view of the contact lens device of FIG. 2.

FIGS. 4A-B are front and cross-sectional views of a contact lens device of a monitoring system, in accordance with some embodiments.

FIGS. 5A-B are front and cross-sectional views of another embodiment of a contact lens device of a monitoring system.

FIGS. 6A-B are front and cross-sectional views of yet another embodiment of a contact lens device of a monitoring system.

FIGS. 7A-B are cross-sectional views of a contact lens device in a nondeformed condition and in a deformed condition.

FIG. 8 is a front view of an alternative embodiment of a contact lens device for use in the monitoring system.

FIG. 9 is a cross-sectional view of the contact lens device of FIG. 8.

FIG. 10 is a partial front view of the contact lens device of FIG. 8.

FIGS. 11A-B are cross-sectional views of the contact lens device of FIG. 8 in a non-deformed condition and in a deformed condition.

FIG. 12 is a headset device of the monitoring system of FIGS. 1A-B.

FIG. 13 is a side view of a portion of the headset device of FIG. 8.

FIG. 14 is a front view of a portion of the head set device of FIG. 8.

FIGS. 15A-B is a partial cross-sectional view of a monitoring system, in accordance with some embodiments.

FIG. 16 is a process diagram for monitoring intraocular pressure, in accordance with some embodiments.

FIG. 17A is a front view of another embodiment of a contact lens device of a monitoring system.

FIG. 17B is a cross-sectional view of the contact lens device of FIG. 17A.

FIGS. 18-19 are partial cross-sectional views of a monitoring system, in accordance with some embodiments.

FIGS. 20A-B are cross-sectional and rear views of another embodiment of a headset device of a monitoring system.

FIG. 21 is a partial cross-sectional view of a monitoring system, in accordance with some embodiments.

FIGS. 22A-B are cross-sectional and rear views of yet another embodiment of a headset device of a monitoring system.

FIGS. 23-24 are partial cross-sectional views of a monitoring system, in accordance with some embodiments.

Like reference symbols in the various drawings indicate like elements.

### DETAILED DESCRIPTION OF ILLUSTRATIVE EMBODIMENTS

Some embodiments a monitoring system 100 may provide a non-invasive technique to measure a patient's intraocular pressure throughout an extended period. For example, the monitoring system 100 may record the intraocular pressure on a regular interval, such as every five minutes, every ten minutes, every twenty minute, every sixty minutes, or more, throughout a period about six hours, about twelve hours, about twenty-four hours, or more. In some circumstances, the intraocular pressure measurements recorded over this period can be used for the diagnosis and management of glaucoma patients and those at risk for glaucoma.

As described in more detail below, the monitoring system 100 may be configured to provide intraocular pressure monitoring in the patient's normal environment without the need to house the patient in a sleep laboratory. Accordingly, the monitoring system 100 may identify patients with large fluctuations in intraocular pressure, or intraocular pressure elevations that occur outside of the ordinary clinical visits. Treatment may then be modified appropriately to limit the deterioration in the condition of the patient's vision.

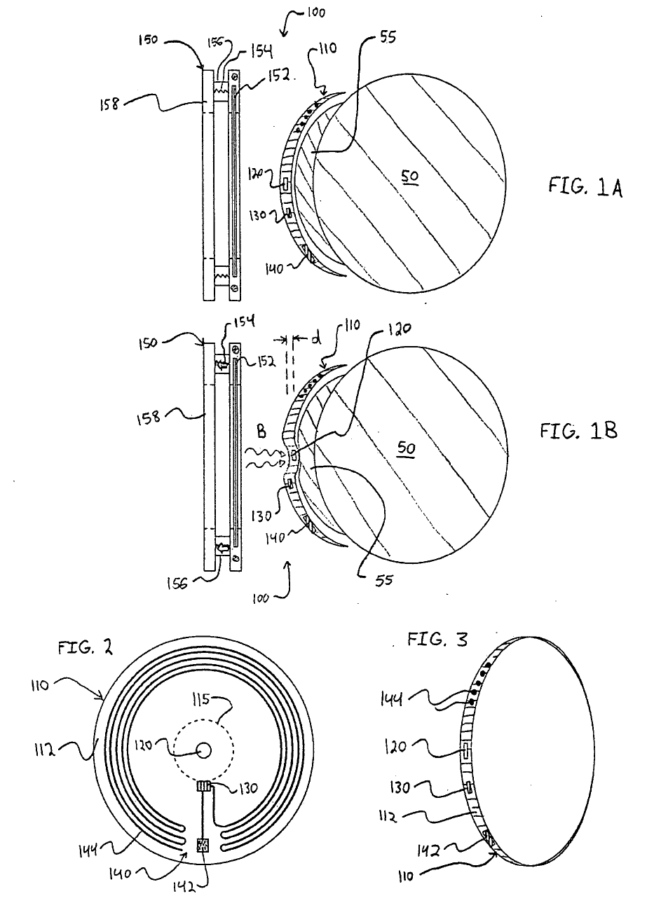

Referring to FIGS. 1A-B, the monitoring system 100 may include a contact lens device 110 that is capable of deflecting in response to a force. The contact lens device 110 can be removably engaged with a patient's eye 50, including the cornea 55. One or more devices are coupled to the contact lens device 110 to cause a portion of the lens device 110 to indent or to indicate when a portion of the lens device 110 has been displaced by a predetermined indentation. For example, a magnet 120 may be embedded in a deformable portion of the contact lens device 110 so that the magnet 120 causes the deformable portion of the contact lens device 110 to be displaced in response to a magnetic field B. In another example, a switch device 130 may be embedded in the contact lens device 110 to indicate when the deformable portion of the contact lens device 110 has been displaced by a predetermined indentation (e.g., refer to displacement d in FIG. 1B). In this embodiment, an antenna device 140 is embedded in the periphery of the contact lens device 110 to wirelessly communicate the indication from the switch device 130.

The monitoring system 100 may also include a headset device 150 that is wearable by the patient. For example, the headset device 150 may be configured in the form of eyeglasses, goggles, or the like. The headset device 150 includes one or more induction coils 152 that generate the magnetic field B to impose a force upon the magnet 120 disposed in the contact lens device 110. As such, an electrical current may be passed through the coils 152 in increasing amounts to apply an increasingly greater force upon the contact lens device 110, thereby causing the deformable portion of the contact lens device 110 to become displaced (refer, for example, to displacement d in FIG. 1B). The headset device 150 may also include at least one force sensor 154 to measure the force applied by the magnetic field B, which causes the force to indent the contact lens device 110 and causes a substantially equivalent force upon the induction coils 152 in the opposite direction. Accordingly, in this embodiment, the force sensor 154 of the monitoring system 100 is coupled to the headset device 150 rather than being embedded in the contact lens device 110. For example, the force sensors 154 can be part of pillar structures 156 that separate the induction coils 152 from the headset frame 158. As described in more detail below, the monitoring system 100 may include a control box 170 in communication with the force sensor (e.g., mounted to the headset or otherwise worn by the patient) so that the force measurement can be stored.

Still referring to FIGS. 1A-B, when the monitoring system 100 is activated to measure the intraocular pressure of the eye 50, electrical current may pass through the induction coil 152, and a repulsive force will be created on the contact lens device 110 (e.g., via the magnetic field B acting upon the magnet 120). This repulsive force causes at least the deformable portion of the lens device 110 (refer to FIG. 1B) to be displaced. As previously described, the magnetic field B also causes a substantially equivalent force in the opposite direction applied the induction coils 152. The force sensors 154, each of which may comprise a strain gauge or the like, measures the force applied by the magnetic field B. The electrical current passing through the induction coils 152 is increased until the switch device 130 coupled to the contact lens device 110 indicates that the predetermined amount of indentation (e.g., refer to displacement d) has been reached. At this point in time, corresponding force measurement (e.g., the strain measurement that is convertible into force measurement) detected by the force sensors 154 are recorded and stored in a memory module (not shown in FIGS. 1A-B). In some embodiments, the control box (not shown in FIGS. 1A-B) may include the control circuitry to convert the force measurement into the intraocular pressure measurement based upon the particular parameters of the monitoring system 100, which is then stored in the memory module. The electrical current will then be shut off and the same process will be repeated in the contralateral eye. The monitoring system 100 may be programmed to reactivate on a regular interval to repeat intraocular pressure tests (e.g., about every five minutes, about every ten minutes, about every twenty minute, about every sixty minutes, or more).

The force measurement data can be transmitted to a computer system. For example, the control box may be connectable to a data port of a personal computer, a handheld computing device, a networked computer system, or the like to transmit the data recorded during the intraocular pressure tests. The connection may include a cable connector or a wireless communication via a RF transceiver device or the like. When the data is received by the computer system, the data may be used for subsequent calculations, for display to a physician, or both. For example, if the data transfer from the control box 170 is in the form of force or strain measurements (e.g., not previously converted into an intraocular pressure measurement), the computer system may be used to convert the force or strain measurement into the intraocular pressure measurement based upon the particular parameters of the monitoring system 100. In another example, the intraocular pressure measurements can be displayed as an intraocular pressure profile showing intraocular pressure measurements as a function of time. Such an intraocular pressure profile can be used at the initial diagnosis stage, when changing a patient's therapy to assess efficacy, or annually to monitor the intraocular pressure control.

Referring now to FIGS. 2-3, some embodiments of the contact lens device 110 may include a soft contact lens 112 comprising a silicone material, a hydrogel material, or another transparent flexible material. The magnet 120 is disposed in the deformable portion 115. For example, the magnet 120 may comprise a small permanent magnet that is embedded in a central portion of the contact lens 112 so that the central portion can indent in response magnetic field B. The magnet 120 may be configured to a size and shape that is suitable for embedded into the contact lens. In one example, the contact lens 112 may have a diameter of about 13 mm to about 16 mm (e.g., about 15 mm in this embodiment), the magnet 120 may have a circular configuration having a diameter of about 0.5 mm to about 3 mm (e.g., about 2 mm in this embodiment), and the deformable portion 115 may have a diameter of about 2 mm to about 6 mm (e.g., about 4 mm in this embodiment). In such embodiments, the predetermined deflection amount d (as shown in FIG. 1B) for the deformable portion 115 may be about 0.1 mm to about 1 mm, about 0.1 mm to about 0.7 mm, or about 0.2 mm to about 0.4 mm (e.g., about 0.3 mm in this particular embodiment). As shown in FIG. 3, the magnet 120 can be thinner than the contact lens 112 so that the magnet 120 is completely encased within the material of the contact lens 112. For example, in some embodiments, the contact lens 112 may have a thickness less than or equal to 1.0 mm, and the magnet 120 may have a thickness of less than or equal to 0.8 mm. The magnet 120 may include electromagnetic materials such as neodymium-iron-boron or other rare earth magnets. In some embodiments, the magnet 120 may comprise a substantially transparent or translucent magnetic material, which may improve the light passage through the visual axis. It should be understood from the description herein that, in other embodiments, the magnet 120 may comprise a ring-shaped permanent magnet with a central opening substantially aligned with the center of the contact lens 112, or the magnet 120 may comprise several smaller magnets place in a circular pattern around the center of the contact lens 112. Such configurations may also provide a substantially clear visual axis.

Still referring to FIGS. 2-3, the antenna device 140 may be coupled to the contact lens 112 to wirelessly communicate when the deformable portion 115 has been displaced by the predetermined amount d (refer to FIG. 1B). For example, at least a portion of the antenna device 140 may be embedded in the periphery of the contact lens 112. In this embodiment, the antenna device 140 comprises a radio-frequency identification (RFID) tag 142 and an antenna line 144 connected to the RFID tag 142. The RFID tag 142 may be a passive radio-frequency identification tag, and the antenna line may be a flexible printed circuit antenna. The antenna line 144 permits communication between the RFID tag 142 embedded in the contact lens 112 and a RFID tag reader incorporated into the headset device 150 (described in more detail below). The antenna line 144 also provides power to the RFID tag 142 using radio waves transmitted from the corresponding antenna line of the tag reader incorporated into the headset device 150. It should be understood from the description herein, that in alternative embodiments, the antenna device 140 may comprise an RFID tag 142 that is self-powered, for example, by a miniature battery or capacitor capable of storing a charge while embedded in the contact lens device 110. In such embodiments, the RFID tag 142 would not require power from the RFID reader disposed on the headset device 150 in order to activate.

The switch device 130 may be disposed proximate to the deformable portion 115 so that the switch device can indicate when the deformable portion 115 has been displaced by the predetermined amount d (refer to FIG. 1B). For example, if the deformable portion has a radius of about 2 mm (a diameter of about 4 mm) about the center of the contact lens device 110, the switch device 130 may be disposed near the junction of the deformable portion 115 at about 2 mm offset from the center of the contact lens device 110. In this embodiment, the switch device 130 comprises a portion of the antenna line 144 that has a break formed into one area. As described in more detail below in connection with FIGS. 7A-B, when the deformable portion 115 of the contact lens device 110 is in a nondeformed condition (refer to FIG. 1A), the opposing ends of the switch device 130 continue to contact one another, thereby permitting the antenna line 144 to wirelessly communicate with the RFID reader incorporated onto the headset device 150. When an intraocular pressure test is activated, the RFID reader on the headset device 150 initiates power to the RFID tag 142, which in turn transmits its unique code back to the RFID reader. When the deformable portion 115 of the contact lens device 110 is displaced to the predetermined amount d (e.g., in the deformed condition shown FIG. 1B), the portion of the antenna line 144 in the switch device 130 is deformed as well. In these circumstances, the opposing ends of the switch device 130 are separated, thereby cutting off the signal from the RFID tag 142. This cut-off of the communication from the antenna line 144 may serve as the indicator to the headset device 150 that the predetermined amount of deformation d has occurred and the intraocular pressure can be measured based upon the force sensor 154 measurement at that point in time.

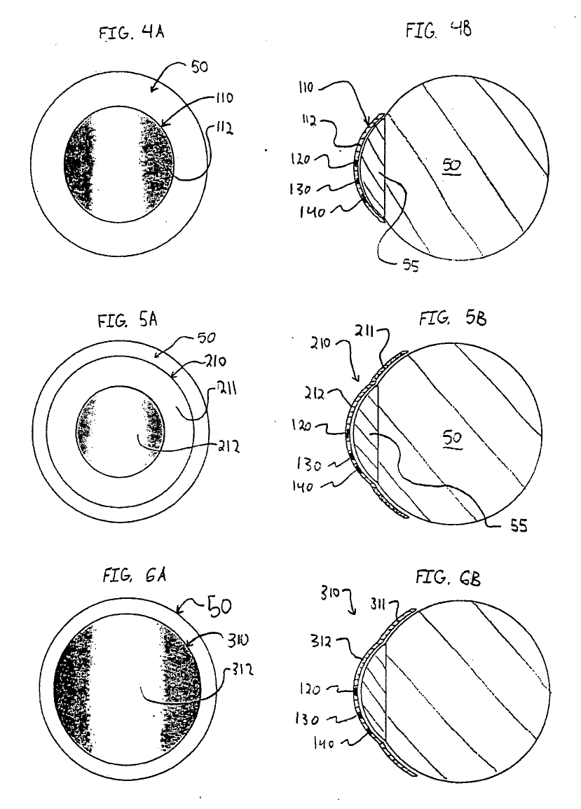

Referring to FIGS. 4A-B, some embodiments of the contact lens device 110 may include the soft contact lens 112 that is configured to removable engage with the cornea 55. As previously described, the soft contact lens 112 comprises a silicone material, a hydrogel material, or another transparent flexible material that is biocompatible with the cornea surface. In this embodiment, the magnet 120, the switch device 130, and the antenna device 140 are embedded in the contact lens 112 during the manufacturing process.

Referring to FIGS. 5A-B, an alternate embodiment of the contact lens device 210 may include a scleral contact lens 210 that has a more rigid ring portion 211 configured to allow the contact lens device 210 to rest on the sclera. In such embodiments, the contact lens device 210 may also include a substantially flexible portion 212 that is disposed over the cornea 55 in a configuration similar to the previously described soft contact lens 112. As previously described, the magnet 120, the switch device 130, and the antenna device 140 may be embedded in the flexible portion 212 during the manufacturing process. In these embodiments, the likelihood of the contact lens device 210 inadvertently moving may be reduced because the contact lens device 210 is generally larger and can be fitted to match the contour change between the sclera and cornea 55. In some circumstances, the reduction in contact lens movement would permit faster pressure measurements by the monitoring system 100.

Referring to FIGS. 6A-B, another alternate embodiment of the contact lens device 310 may include a scleral contact lens 312 made entirely of flexible material, such as a silicone material, a hydrogel material, or another transparent flexible material that is biocompatible with the cornea surface. The contact lens device 310 includes a ring portion 311 configured to allow the contact lens device 310 to rest on the sclera and a central portion 312 that is disposed over the cornea 55. As previously described, the magnet 120, the switch device 130, and the antenna device 140 may be embedded in the scleral contact lens 312 during the manufacturing process. Again, the likelihood of the contact lens device 310 inadvertently moving may be reduced because the contact lens device 310 is generally larger and can be fitted to match the contour change between the sclera and cornea 55. Such a reduction in contact lens movement may permit faster pressure measurements by the monitoring system 100.

Referring to FIGS. 7A-B, the switch device 130 may comprise a mechanical switch that is adjusted from a first configuration (e.g., FIG. 7A) to a second configuration (e.g., FIG. 7B) when the deformable portion 115 has been displaced by the predetermined amount d (refer also to FIG. 1B). As previously described in connection with FIGS. 2-3, some embodiments of the switch device 130 comprise a portion of the antenna line 144 that has a break 135 formed into one portion thereof. As such, when the deformable portion 115 of the contact lens device 110 is in a nondeformed condition (refer to FIG. 7A), the opposing ends 134a and 134b of the switch device 130 continue to contact one another, thereby permitting the antenna line 144 to wirelessly communicate with the RFID reader incorporated onto the headset device 150. When an intraocular pressure test is activated, the RFID reader on the headset device 150 initiates power to the RFID tag 142, which in turn transmits its unique code back to the RFID reader. When the deformable portion 115 of the contact lens device 110 is displaced to the predetermined amount d (refer to FIG. 7B), the portion of the antenna line 144 in the switch device 130 is deformed as well. In these circumstances, the opposing ends 134a and 134b of the switch device 130 are separated at the break 135, thereby cutting off the signal from the RFID tag 142. This cut-off in the communication from the antenna line 144 may serve as the indicator to the RFID reader on the headset device 150 that the predetermined amount of deformation d has occurred and the intraocular pressure can be measured based upon the force sensor 154 measurement at that point in time (as described in more detail below).

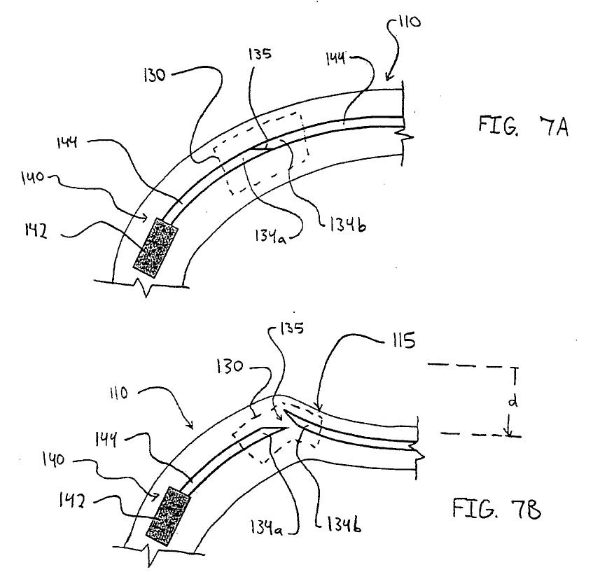

Some alternative embodiments of the contact lens device may include a switch device other than the individual break 135 in the antenna line 144 as described in connection with FIGS. 7A-B. For example, as described in connection with FIGS. 8-10 and 11A-B, some embodiments of the contact lens device 410 may include a switch device 430 having a number of opposing contacts that are adjustable to open and close the antenna line from the RFID tag 142.

Referring to FIGS. 8-10 and 11A-B, the contact lens device 410 is capable of deflecting in response to a force and can be used in the monitoring system 100 (similar to previously described embodiments). As such, the contact lens device 410 can be used with the headset device 150 that is in a wearable form, such as in the form of eyeglasses, goggles, or the like (refer, for example, to FIGS. 12-14). Similar to embodiments previously described in connection with FIGS. 1A-B, the headset device 150 includes one or more induction coils 152 that generate the magnetic field B to impose a force upon the magnet 120 disposed in the contact lens device 410. When an electrical current is passed through the coils 152 (FIGS. 1A-B) in increasing amounts to apply an increasingly greater force upon the contact lens device 410, the deformable portion 415 of the contact lens device 410 can be displaced (refer, for example, to displacement d₂ in FIG. 11B). As previously described, the headset device 150 may also include at least one force sensor 154 (FIGS. 1A-B) coupled to the headset device 150 rather than being embedded in the contact lens device 410.

Referring now to FIGS. 8-10, the contact lens device 410 includes a deformable portion 415 that is at least partially defined by groove 417. The groove 417 may provide a region of reduced thickness that facilitates local deflection when a force is applied to the deformable portion 415 (e.g., when the magnet 120 reacts to a magnetic field). In this embodiment, groove 417 may be formed as a score line extending circumferentially about a central axis of the contact lens device 410. In such circumstances, the depth of the groove 417 can range from about 10% to about 90% of the contact lens thickness depending of the type of contact lens material, the deflection displacement d₂, and other factors. Also, in these embodiments, the width of the groove may be about 100 μm or less (preferably about 10 μm to about 90 μm) depending of the type of contact lens material, the deflection displacement d₂, and other factors.

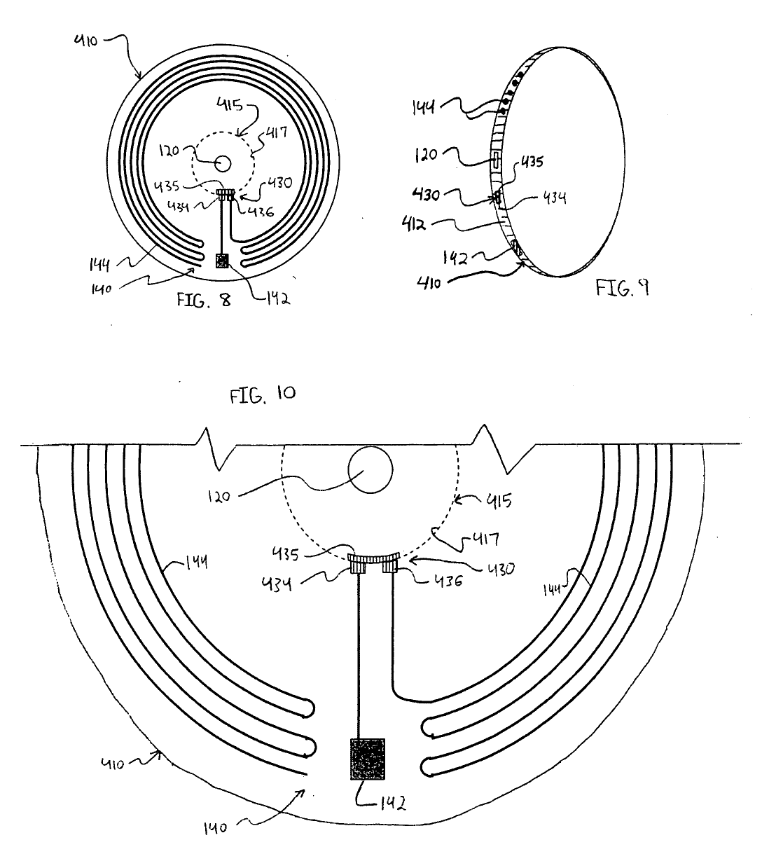

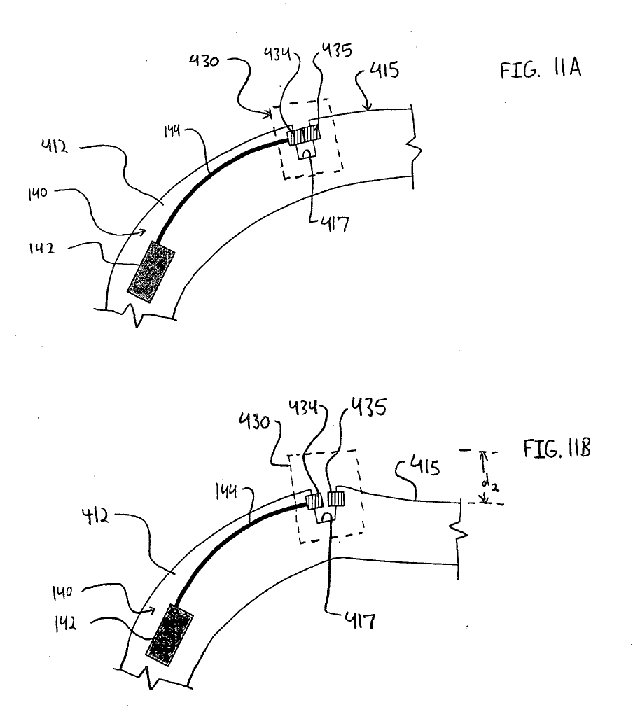

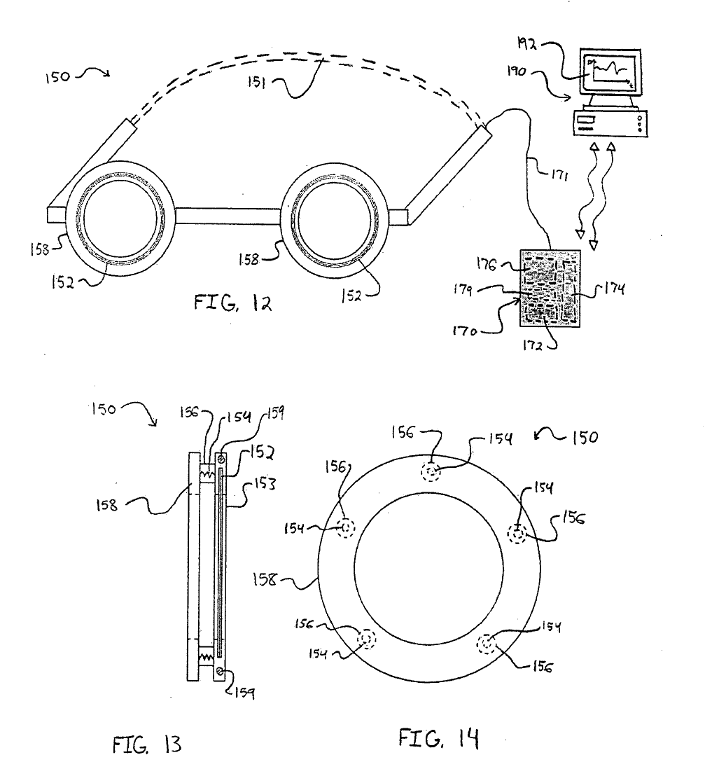

As shown in FIG. 10, the switch device 430 of the contact lens device 410 includes three electrically conductive elements 434, 435, and 436 arranged proximate to the groove 417 of the deformable portion 415. In this embodiment, the first conductive element 435 has a greater size such that it is configured to engage both the first and third contact elements 434 and 436. For example, the second conductive element 435 can be arranged within the deformable portion 415 of the contact lens device 410, and the first and third elements 434 and 436 can be arranged on the opposite side of the groove 417. The first conductive element 434 is connected to the RFID tag 142, while the third conductive element 436 is connected to the antenna 144. Accordingly, the RFID tag 142 can be connected to the associated antenna 144 when both of the first and third elements 434 and 436 are in contact with the intermediate element 435.

In use, the switch device 130 can operate such that the first and third elements 434 and 436 both engage the second element 435 when the contact lens device 410 is in the non-deformed state (described in more detail below in connection with FIGS. 11A-B). When the deformable portion 415 of the contact lens device 110 is in the non-deformed condition (refer to FIG. 11A), the antenna line 144 can wirelessly communicate with the RFID reader incorporated onto the headset device 150. When an intraocular pressure test is activated, the RFID reader on the headset device 150 initiates power to the RFID tag 142, which in turn transmits its unique code back to the RFID reader. When the deformable portion 415 of the contact lens device 410 is deformed to a deflection displacement d₂ (e.g., the deformed state described in connection with FIG. 11B), a gap can be formed across the groove 417 to thereby separate the contact between the elements 434, 435, and 436. In these circumstances, the RFID tag 142 is disconnected from the antenna 144, thereby cutting off the signal from the RFID tag 142. This cut-off of the communication from the antenna line 144 may serve as the indicator to the headset device 150 that the predetermined amount of deformation d₂ has occurred and the intraocular pressure can be measured based upon the force sensor 154 measurement at that point in time. Such embodiments of the switch device 430 for the contact lens device may provide a reliable and accurate process for determining the deformation of the contact lens device 410. Furthermore, in this embodiment, no portion of the RFID antenna 144 or tag 142 is necessarily within the zone of deformation in the contact lens device 410.

Still referring to FIGS. 8-10, this embodiment of the contact lens device 410 may include a soft contact lens 412 comprising a silicone material, a hydrogel material, or another transparent flexible material. Similar to previously described embodiments, the magnet 120 may comprise a small permanent magnet that is embedded in a central portion of the contact lens 112 so that the central portion can indent in response magnetic field B. The magnet 120 may be configured to a size and shape that is suitable for embedded into the contact lens. In one example, the contact lens 112 may have a diameter of about 13 mm to about 16 mm (e.g., about 15 mm in this embodiment), the magnet 120 may have a circular configuration having a diameter of about 0.5 mm to about 3 mm (e.g., about 2 mm in this embodiment), and the deformable portion 415 may have a diameter of about 2 mm to about 6 mm (e.g., about 4 mm in this embodiment). In such embodiments, the predetermined deflection amount d (as shown in FIG. 1B) for the deformable portion 115 may be about 0.1 mm to about 1 mm, about 0.1 mm to about 0.7 mm, or about 0.2 mm to about 0.4 mm (e.g., about 0.3 mm in this particular embodiment). The size of the deformable portion 415 can be slightly larger than the permanent magnet 120 in the contact lens device 410. For example, if the magnet 120 comprises a permanent magnetic having a diameter of about a 2 mm magnet, and the deformable portion 410 can be defined by the groove 417 having a diameter of about 3 mm to about 4 mm.

Similar to previously described embodiments, the magnet 120 can be thinner than the contact lens 412 so that the magnet 120 is completely encased within the material of the contact lens 412 (as shown, for example, in FIG. 9). For example, in some embodiments, the contact lens 112 may have a thickness less than or equal to 1.0 mm, and the magnet 120 may have a thickness of less than or equal to 0.8 mm. The magnet 120 may include electromagnetic materials such as neodymium-iron-boron or other rare earth magnets. In some embodiments, the magnet 120 may comprise a substantially transparent or translucent magnetic material, which may improve the light passage through the visual axis. It should be understood from the description herein that, in other embodiments, the magnet 120 may comprise a ring-shaped permanent magnet with a central opening substantially aligned with the center of the contact lens 412, or the magnet 120 may comprise several smaller magnets place in a circular pattern around the center of the contact lens 412. Such configurations may also provide a substantially clear visual axis.

Still referring to FIGS. 8-10, the antenna device 140 may be coupled to the contact lens 412 to wirelessly communicate when the deformable portion 415 has been displaced by the predetermined amount d₂ (refer to FIG. 11B). For example, at least a portion of the antenna device 140 may be embedded in the periphery of the contact lens 412. In this embodiment, the antenna device 140 comprises the RFID tag 142 and the antenna line 144 that is connectable to the RFID tag 142. Similar to previously described embodiments, the antenna line 144 permits communication between the RFID tag 142 embedded in the contact lens 112 and a RFID tag reader incorporated into the headset device 150. The antenna line 144 also provides power to the RFID tag 142 using radio waves transmitted from the corresponding antenna line of the tag reader incorporated into the headset device 150.

Referring now to FIGS. 11A-B, the switch device 430 may comprise one or more adjustable contact elements that shift from a first configuration (e.g., FIG. 11A) to a second configuration (e.g., FIG. 11B) when the deformable portion 415 has been displaced by the predetermined amount d₂ (refer also to FIG. 11B). As previously described in connection with FIGS. 8-10, some embodiments of the switch device 430 comprise three electrically conductive elements 434, 435, and 436 (element 436 not shown in this view) arranged along to the groove 417 that at least partially defines the deformable portion 415. As such, when the deformable portion 415 of the contact lens device 110 is in a non-deformed condition (refer to FIG. 11A), the first and third elements 434 and 436 of the switch device 130 continue to contact the intermediate element 435, thereby permitting the antenna line 144 to wirelessly communicate with the RFID reader incorporated onto the headset device 150. When an intraocular pressure test is activated, the RFID reader on the headset device 150 initiates power to the RFID tag 142, which in turn transmits its unique code back to the RFID reader. When the deformable portion 415 of the contact lens device 110 is displaced to the predetermined amount d₂ (refer to FIG. 11B), the switch device 430 is deformed as well. In particular, the first and third elements 434 and 464 are separated from the intermediate element 435, thereby cutting off the signal from the RFID tag 142 (e.g., the winding portion of the antenna 144 is no longer in communication with the RFID tag 142). This cut-off in the communication from the antenna line 144 may serve as the indicator to the RFID reader on the headset device 150 that the predetermined amount of deformation d₂ has occurred and the intraocular pressure can be measured based upon the force sensor 154 measurement at that point in time (as described in more detail below).

Referring to FIGS. 12-14, the headset device 150 of the monitoring system 100 may be configured as eyeglasses or goggles that are wearable by the user. The headset device 150 can be worn contemporaneously with at least one contact lens device 110 (or with any of the alternative contact lens devices 210, 310, and 410) so as to monitor the intraocular pressure on a repeated basis over an extended period, such as a 6-hour period, a 12-hour period, a 24-hour period, or more. In some embodiments, the headset device 150 can be maintained in a substantially stationary position relative to the user's head using a headband 151 that wraps around a portion of the user's head. The headset device 150 includes a frame 158 that arranges the induction coils 152 in an orientation proximate to the contact lens device 110 disposed in the user's eye. As shown in FIG. 12, the frame 158 may provide alignment with the induction coils 152 over each of the user's eyes so that the user can wear a contact lens device 110 in each eye and the associated induction coils 152 are arranged proximate thereto. In some embodiments, the frame 158 may also include prescription lenses or the like that aid in the user's visual focus, thereby temporarily replacing the eyeglasses ordinary worn by the user. The induction coils 152 may be embedded in or otherwise coupled to a support plate 153, which is mounted to the frame 158 via one or more pillar structures 156. The support plate 153 may be in the form of a ring (e.g., having a central opening therethrough), and the support plate 153 may comprise a material having ferromagnetic properties that enhance the magnetic field generated by the induction coils 152. It should be understood that, in some embodiments, a sufficiently strong magnetic field can be generated induction coils 152 even if the support plate 153 comprises an electrically insulating material such as a moldable plastic. The force sensors 154 (e.g., strain gauges or the like) may be integrated into the pillar structures 156 so that a force urging the induction coils 152 or support plate 153 toward the frame 158 can be detected by the force sensors 154. Although the embodiment depicted in FIG. 13 shows the induction coils 152 arranged in a substantially axially aligned orientation, it should be understood that other embodiments of the headset device 150 may utilize multiple independent induction coils 152 arranged at different axial angles to provide a substantially perpendicular force to the contact lens device 110 even when the user's eye is directed toward an upward, downward, or sideways direction.

As previously described, the headset device 150 may include a headset antenna line 159 that wireless communicates with the antenna line 144 (FIG. 2) of the contact lens device 110. The headset antenna line 159 may be coupled to the support plate 153 so that the headset antenna line 159 is oriented proximate to the contact lens device 110. The headset antenna line may be in electrical communication with a reader device 179 disposed in the control box 170. Alternatively, the reader device 179 may be embedded or otherwise incorporated into the frame 158 of the headset device 150. In some embodiments, the reader device may comprise an RFID reader 179 that is configured to wirelessly communicate with an RFID tag 142 (FIG. 2) via the antenna line 144 (FIG. 2) and the headset antenna line 159.

Still referring to FIGS. 12-14, the monitoring system 100 may include a control box 170 in communication with the induction coil 152, the force sensors 154, and other electronic circuits incorporated onto the headset device 150. In this embodiment, the control box 170 is configured to be worn by the user in a location other than the user's head (e.g., attached to a waist band or retained in a pocket). The control box 170 can be electrically connected to the components disposed on the headset device 150 via at least one wire 171. In other embodiments, the control box 170 may be mounted into the frame of the headset device 150 (e.g., housed in a curved frame portion positioned proximate the user's ear). The control box 170 may include a controller circuit 172 that controls the activation and pressure sensing operations of the monitoring system. As such, the monitoring system 100 can provide passive measurements (e.g., no required activation step by the patient), so the system 100 is capable of monitoring the intraocular pressure even when the patient is asleep. Also, the control box may include a power source 174, such as a rechargeable battery or the like, that provides electrical power to the induction coils 152, the force sensors 154, and other components of the headset device 150. A memory module 176 may be disposed in the control box 170 so that data such as dates, times, force measurements from the force sensors 154, and the like, can be recorded over an extended period while the monitoring system 100 is worn by the user.

As previously described, the force or pressure data and other data can be transmitted from the control box 170 to a computer system 190 (FIG. 12). When the data is received by the computer system 190, the data may be used for subsequent calculations, for display to a physician, or both. For example, if the data transfer from the control box 170 is in the form of force or strain measurements (e.g., not previously converted into an intraocular pressure measurement), the computer system may be used to convert the force or strain measurement into the intraocular pressure measurement based upon the particular parameters of the monitoring system 100. In another example, the intraocular pressure measurements can be displayed as an intraocular pressure profile 192 showing intraocular pressure measurements as a function of time. Such an intraocular pressure profile 192 can be used at the initial diagnosis stage, when changing a patient's therapy to assess efficacy, or annually to monitor the intraocular pressure control.

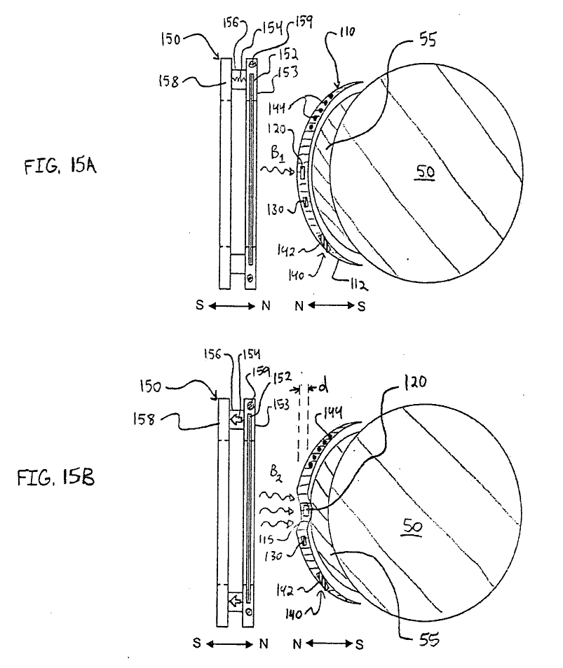

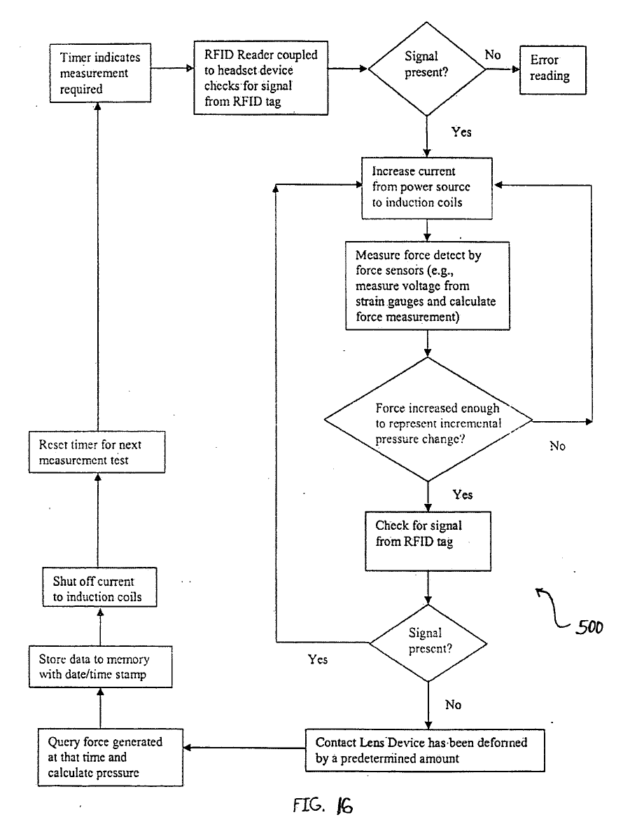

Referring now to FIGS. 15A-B, the monitoring system 100 may be configured to increase the magnetic field (refer to B₁ and B₂) until the force applied to the contact lens device 110 causes the deformable portion to be displaced by a predetermined amount d. In some embodiments, the control box 170 (FIG. 12) may include circuitry to activate the monitoring system 100 to measure the intraocular pressure of the eye 50 at regular intervals (e.g., about every 5 minutes, about every, 10 minutes, about every 20 minutes, about every 60 minutes, or more). As shown in FIG. 15A, when the monitoring system 100 is activated to measure the intraocular pressure, electrical current from the power source 174 (FIG. 12) may pass through the induction coil 152, thereby creating a magnetic field B₁. The magnetic field B₁ causes a repulsive force to act upon on the magnet 120 of contact lens device 110. A substantially equivalent force in the opposite direction will also act upon the induction coils 152 (and the support plate 153 carrying the induction coils 152), and the force sensors 154 are arranged to detect this force. In these embodiments, the magnetic force may not be affected by the user's eyelids. In such circumstances, the monitoring system 100 is capable of monitoring the intraocular pressure regardless of whether the user's eyelids are opened or closed.

As shown in FIG. 15B, if the magnetic field B₁ is not stronger enough to deform the contract lens device 110 by a predetermined amount d, the circuitry of the control box 170 may increase the current through the induction coils 152 so that a greater magnetic field B₂ is generated. The greater magnetic field B₂ causes a greater force to act upon on the magnet 120 of contact lens device 110, until the deformable portion 115 of the contact lens device 110 is displaced by a predetermined amount d. The force sensors 154 detect this greater force generated by the magnetic field B₂, as previously described. The switch device 130 disposed of the contact lens device 110 indicates when the deformable portion 115 is displaced by the predetermined amount d, for example, by cutting-off antenna communication between the antenna device 140 and the headset device 150. At this point in time, corresponding force measurement (e.g., the strain measurement signal that can be converted into a force measurement) is detected by the force sensors 154. The force measurements or the like are recorded and stored in the memory module 176 of the control box. The control circuitry 172 of the control box 170 may then shut off the electrical current to the induction coils 152, and the same process will be repeated in the contralateral eye (if another contract lens device 110 is disposed in the contralateral eye). The circuitry of the control box 170 may be programmed to reactivate the monitoring system 100 on a regular interval to repeat these intraocular pressure tests. Accordingly, in some embodiments, the monitoring system 100 can provide measurement of intraocular pressure throughout a 24-hour period without the need for the patient to be kept in a hospital or in a sleep laboratory (e.g., a patient may be able to continue normal activities while the intraocular pressure monitoring system 100 is operational).

In the embodiment depicted in FIGS. 15A-B, the switch device 130 may cut-off communication between the RFID tag 142 and the RFID reader 179 (e.g., disposed in the control box 170 or incorporated into the frame 158) to indicate when the predetermined indentation d has occurred. When the monitoring system 100 is activated, the RFID reader 179 initiates power to the RFID tag 142 (e.g., via the wireless communication between the antenna line 144 and the headset antenna line 159), which in turn transmits its unique code back to the RFID reader 179. When the deformable portion 115 of the contact lens device 110 is displaced to the predetermined amount d (refer to FIG. 15B), a portion of the antenna line 144 in the switch device 130 is deformed as well, thereby cutting off the signal from the RFID tag 142. This cut-off in the communication from the antenna line 144 may serve as the indicator to the RFID reader 179 on the headset device 150 that the predetermined amount of deformation d has occurred and the intraocular pressure can be measured based upon the force sensor 154 measurement at that point in time. An exemplary process 500 for the measuring the pressure is described in connection with FIG. 16. In this embodiment, circuitry in the control box 170 will increase the current through the induction coils 152 so that the force acting upon the contact lens device 110 will increase in selected increments. These increments could be set to correspond to different pressure increments. After each incremental increase in force, the RFID tag 142 will be signaled to broadcast its code via the antenna line 144 to the headset antenna line 159. When the cornea has been indented by the correct amount and the switch device 130 has been triggered, the RFID tag 142 would stop broadcasting. As such, the amount of force at this point in time can be measured by the force sensors 154 and the corresponding pressure can be calculated and recorded in the memory module 176.

As previously described, in some embodiments the force measurements from the force sensors 154 on the headset device 150 can be transmitted to a computer system 190 for subsequent calculations or processing. Alternatively, the force measurements can be processed by the control box 170 to convert the data into intraocular pressure measurements before the data is transmitted to the computer system 190. In one example, the intraocular pressure can be calculated based on the measured amount of force to produce a fixed amount of indentation. Such a calculation for the intraocular pressure may be similar to the principal of the Schiotz indentation tonometer, except that the monitoring system 100 described herein can indent the eye 50 a predetermined amount while varying the force applied to the eye 50. Accordingly, the monitoring system 100 may provide a more accurate and reproducible intraocular pressure measurement.

It should be understood that other embodiments of the headset device 150 may utilize multiple independent induction coils 152 arranged at different axial angles to allow measurement of intraocular pressure with the eye 50 in various positions. One exemplary configuration would employ nine induction coils 152: one in primary forward position (e.g., in substantial axial alignment with the contact lens device 110 when the eyes are directed straight ahead) and one in each of the other eight positions of gaze (up, down, right, left, up and right, up and left, down and right, down and left). The control box 170 may be configured to selectively apply current to the nine induction coils depending on the orientation of the user's eye 50. For example, the selected induction coil 152 could be determined by initially activating all the coils 152. Additional force sensors (e.g., strain gauges or the like) can be integrated with the pillar structures 156 to measure the tangential forces. The coil 152 that registers the least amount of tangential force would be the one closest to perpendicular direction of the contact lens device 110. All the other coils 152 may then be shut down and the magnetic field B would be generated using the selected coil 152. The pressure measurement may be measured as previously described, for example, in FIG. 16. Alternatively, each of the nine coils could be activated sequentially at a fixed sequence until the coil 152 with the smallest tangential force is found. The intraocular pressure would then be measured using that coil. All the other coils 152 may then be shut down and the magnetic field B would be generated using the selected coil 152. The pressure measurement may be measured as previously described, for example, in FIG. 16.

In another alternative embodiment, one of the nine inductive coils 152 can be selected using one or more scleral search coils. For example, an electrically conducting coil would be placed in the periphery of the contact lens (the “scleral search coil”). Two of the induction coils 152 of the headset device 150 with axes placed 90-degrees apart would generate alternating magnetic fields. This can induce an alternating voltage in the scleral search coil proportional to the sine of the horizontal and vertical eye position. The voltage can be measured directly on the contact lens device 110 as part of the RFID tag functionality, or can be transmitted back to the headset device 150 for measurement using a wire coupled to the contact lens device 110. Once the eye position is known, the appropriate induction coil 152 on the headset device 150 can be activated to measure the intraocular pressure.

In some circumstances, the amount of force that can be generated to indent the contact lens device 110 as described in a connection with FIG. 15B can be approximated from the Imbert-Fick principle. Assuming that the cornea of the human eye is an infinitely thin spherical surface, the amount of force required to applanate or flatten an area of the cornea equal to the size of the indentation disc is given by:

F=P_{eye}A_{disc}

where P_{eye} is the intraocular pressure, and A_{disc} is the surface area of the indentation disc. For purposes of this example, the intraocular pressure would range between 0 mmHg and 40 mmHg. Accordingly, if the predetermined deformation amount causes an indentation with a disc of about 2 mm diameter, a force of about 16.75×10⁻³ N would be required.

The induction coils 152 can be configured based on a number of factors. For example, the number of turns of wire in the induction coil 152 and the electrical current required to generate the force is dependent on the type of permanent magnet, shape, size and distance. In some circumstances, approximations can be made using certain physical equations. For example, the force between two magnets can be estimated by:

$$F = \frac{1}{{μ}_{media}} {B}_{1} {B}_{2} {A}_{pole}$$

where B₁ is the flux density of the first magnet, B₂ is the flux density of the second magnet, Aₚₒₗₑ is the pole area, and μ_{media} is the permeability of the medium between the 2 magnets. In these circumstances, the flux density of a disc shaped permanent magnet can be approximated as:

$${B}_{1} = \frac{{B}_{r}}{2} [ \frac{t + x}{\sqrt{{R}^{2} + {( t + x )}^{2}}} - \frac{x}{\sqrt{{R}^{2} + {x}^{2}}} ]$$

where Bᵣ is the residual flux density of the permanent magnet, t is the thickness of the magnet, R is the radius, and x is the distance from the surface of the magnet. Also in these circumstances, the flux density of the induction coil can be approximated from solenoid equations:

$${B}_{2} = \frac{{μ}_{0} I \cdot N}{L}$$

where μ₀ is the permeability of free space or air, I is the electric current, N is the number of coil turns, and L is the length of the solenoid. With this equation, one may approximate the number of turns for the induction coil 152 and the electrical current passing through the induction coil 152 required to generate the force needed to measure the maximum expected intraocular pressure. While this equation may be strictly valid only for calculation of the flux density at the center of a long solenoid, it is believed that the equation gives a reasonable approximation because the induction coil 152 may be an open ring which can be placed proximate to the wearer's orbital rim so that the surface of the eye and contact lens device 110 is within or just outside of the induction coil 152.

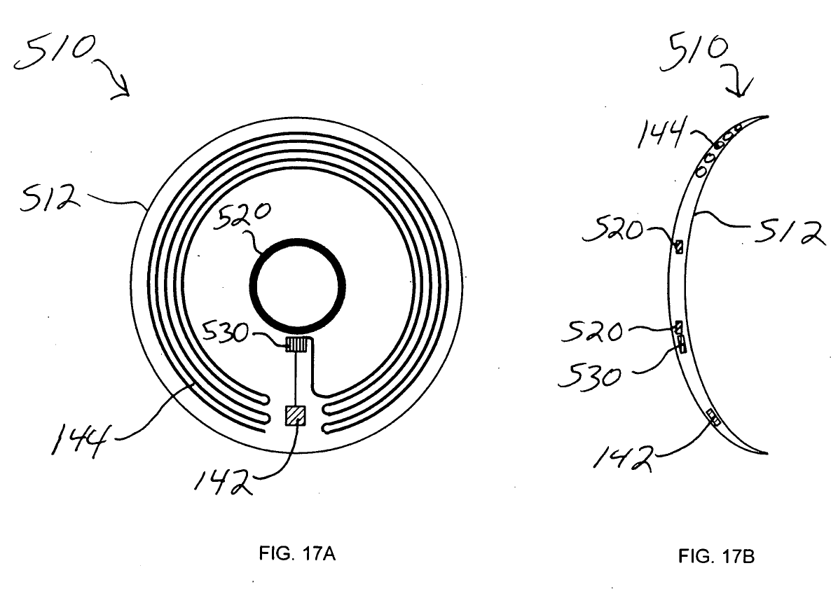

Referring to FIGS. 17-24, some embodiments of a monitoring system may include a contact lens device 510 that is capable of causing the cornea of a user to match the inner contour of the contact lens device 510, which can be used to perform dynamic contour tonometry. For example, the contact lens device 510 may include a contact lens 512 comprising a generally rigid material such as fluorosilicon acrylate or another rigid gas permeable (RGP) lens material. In this embodiment, the lens body is formed from the generally rigid material. In alternative embodiments, the lens device may include a generally rigid central portion (with the magnet 520 and pressure sensor 530 embedded therein as described below) surrounded by a generally flexible skirt to provide enhanced comfort to the user.

silicone material, a hydrogel material, or another transparent flexible material. One or more devices can be coupled to the contact lens device 510 to provide a force to the contact lens device 510, causing at least a portion of the patient's cornea 55 to conform to the inner contour the lens device 510. When the contour of the engaged portion of the cornea 55 substantially matches the inner contour of the lens device 510, the patient's intraocular pressure can be measured by detecting the tear film pressure along the surface of the cornea (described in greater detail below). In some circumstances, the monitoring system 500 may record the intraocular pressure on a regular interval (e.g., every five minutes, every ten minutes, every twenty minute, every sixty minutes, or more, throughout a period about six hours, about twelve hours, about twenty-four hours, or more) and these recorded pressures can be used for the diagnosis and management of glaucoma patients and those at risk for glaucoma. As described previously, the monitoring system 500 may be configured to provide intraocular pressure monitoring in the patient's normal environment without the need to house the patient in a sleep laboratory.

Referring now to FIGS. 17A-B, the contact lens device 510 can include one or more magnets 520 that may be embedded in a portion of the contact lens device 510 so that the contact lens device 510 can mildly deform the cornea 55 in response to a magnetic field, until the curvature of a portion of the cornea 55 substantially matches the contour of an abutting portion of the contact lens device 510. As previously described, in those embodiments in which the lens body comprises a generally rigid material, the magnet 520 (and pressure sensor 530 described below) may be embedded in the generally rigid region of the lens device 510. In some embodiments, the magnet 520 may comprise an annular-shaped permanent magnet that is embedded around a central portion of the contact lens 512. The magnet 520 may be configured to a size and shape that is suitable for embedding into the contact lens 512. In one example, the contact lens 512 may have a diameter of about 13 mm to about 16 mm (e.g., about 15 mm in this embodiment), the magnet 520 may have an annular configuration having an outside diameter of about 3 mm to about 5 mm (e.g., about 4 mm in this embodiment) and an inside diameter of about 2.5 mm to about 4.5 mm (e.g., about 3.5 mm in this embodiment). As shown in FIG. 17B, the magnet 520 can be thinner than the contact lens 512 so that the magnet 520 is completely encased within the material of the contact lens 512. For example, in some embodiments, the contact lens 512 may have a thickness less than or equal to 1.0 mm, and the magnet 520 may have a thickness of less than or equal to 0.8 mm. As in previous embodiments, the magnet 520 may include electromagnetic materials such as neodymium-iron-boron or other rare earth magnets. In some embodiments, the magnet 520 may comprise a substantially transparent or translucent magnetic material, which may improve the light passage through the visual axis. It should be understood from the description herein that, in other embodiments, the magnet 520 may comprise a small permanent magnet that is embedded in a central portion of the contact lens 512 (e.g., similar to the permanent magnet 120 described previously in connection with FIGS. 2-3).

Similar to previously described embodiments, the contact lens device 510 can include an antenna 144 that enables wireless communication to other components in the system (such as the headset or control module). In this embodiment, the contact lens device 510 includes a pressure sensor 530 (e.g., a capacitive pressure sensor or the like) that can detect pressure along the tear-film layer (described in more detail in connection with FIG. 19). The pressure information from the pressure sensor 530 can be communicated from the antenna 144 to a corresponding antenna on the headset device for storage and subsequent analysis. Optionally, the contact lens device 510 may include a device 142 (e.g., RFID device, or another supporting component for the pressure sensor 530) that facilitates the wireless communication from the antenna 144.

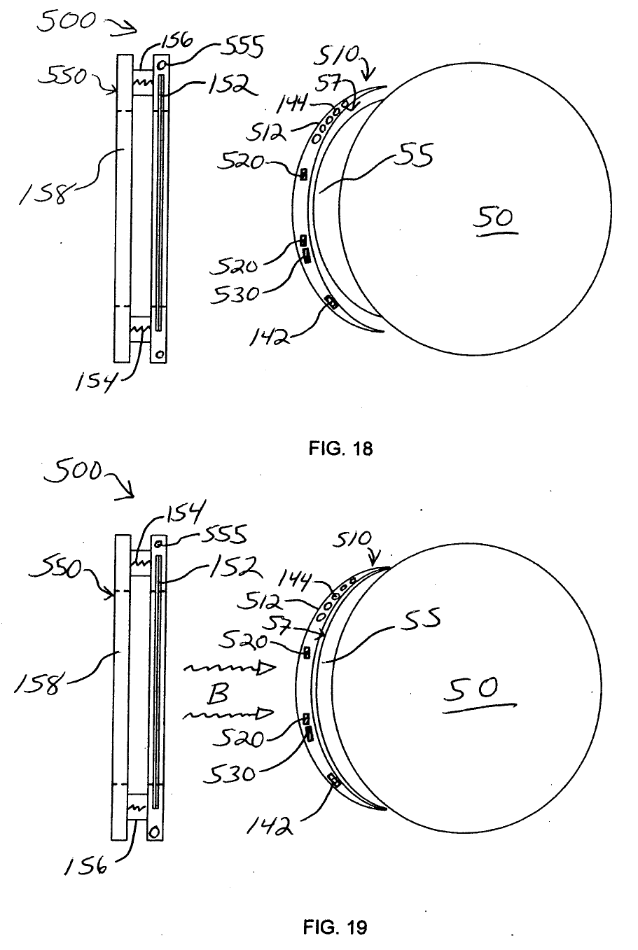

Referring now to FIGS. 18-19, in some embodiments, the magnet 520 can be used to apply a force to the cornea 55 so that the cornea 55 substantially matches the inner contour of the contact lens device 510. When these contours are substantially equivalent, the intraocular pressure and the pressure of a tear-film layer 57 (e.g., located between the cornea 55 and the contact lens device 510) are substantially equal. (The tear-film layer 57 depicted in FIGS. 18-19 is exaggerated for purposes of illustration.) In these circumstances, a pressure sensor 530 embedded in the contact lens device 510 can measure the pressure in the tear-film layer 57. The lens device 510 can also include the RFID tag 142 and the antenna device 140, embedded in the periphery of the contact lens device 510, to wirelessly communicate information detected the pressure sensor 530.

Referring now to FIG. 18, the monitoring system 500 may also include a headset device 550 that is wearable by the patient. For example, the headset device 550 may be configured in the form of eyeglasses, goggles, or the like. The headset device 550 includes the one or more induction coils 152 that generate a magnetic field B to impose a force upon the magnet 520 disposed in the contact lens device 510. As such, an electrical current may be passed through the coils 152 in increasing amounts to apply an increasingly greater force upon the contact lens device 510, thereby causing the cornea 55 to slightly deform such that the contour of at least a portion of the cornea 55 substantially matches the inner contour of the contact lens device 510.

The headset device 550 may also include the at least one force sensor 154 to measure the force applied by the magnetic field B, which causes the contact lens device 510 to abut and press against the cornea 55 with enough force to slightly deform the cornea 55 and also causes a substantially equivalent reaction force upon the induction coils 152 in the opposite direction. For example, the force sensors 154 can be part of the pillar structures 156 that separate the induction coils 152 from the headset frame 158. As described in more detail above in connection with FIG. 12, the monitoring system 500 may include the control box 170 in communication with the force sensor 154 (e.g., mounted to the headset or otherwise worn by the patient). Information from the force sensor 154 can be used to verify that the correct amount of pressure is being applied by the headset 150 (via the magnetic field B) to the contact lens device 510 and to modify the strength of the magnetic field B (e.g., using the induction coil 152) until a predetermined force is applied. For example, in some embodiments, the magnetic field B can be controlled so that the resulting force applied from the magnet 520 to the cornea 55 is about 0.5 grams to about 1.4 grams and about 1 gram to about 3 grams in particular embodiments. Such a force can be used to provide contour matching between the contact lens device 510 and the cornea 55 without substantial deformation of the cornea 55.

Referring now to FIG. 19, when the monitoring system 500 is activated to measure the intraocular pressure of the eye 50, electrical current may pass through the induction coil 152, and a repulsive force will be created on the contact lens device 510 (e.g., via the magnetic field B acting upon the magnet 520). This repulsive force causes the contact lens device 510 to abut and press against the cornea 55, causing at least a portion of the cornea 55 to slightly deform to match the inner contour of the contact lens device 510. As previously described, the magnetic field B also causes a substantially equivalent force in the opposite direction applied the induction coils 152. The force sensors 154, each of which may comprise a strain gauge or the like, measures the force applied by the magnetic field B. The electrical current passing through the induction coils 152 is increased until the force sensor 154 indicates that the predetermined amount of force (e.g., between about 9.8 and 39.4 mN, about 12 mN, about 31.7 mN, or the like) has been reached. At this amount of force, the cornea 55 may have a curvature that substantially matches the inner contour of an abutting portion of the contact lens device 510. When this condition occurs, the pressure on either side of the cornea 55 (e.g., the intraocular pressure and the pressure of the tear-film layer 57) may be substantially equivalent. Thus the intraocular pressure can be determined by measuring the pressure of the tear-film layer 57 using the pressure sensor 530 (e.g., a capacitive pressure sensor or the like). Information output from the pressure sensor 530 can be communicated via the antenna 144 to the corresponding antenna 555 on the headset device 550 for storage in a memory module 176 (previously described in connection with FIG. 12). In some embodiments, the control box may include the control circuitry to convert the information from the pressure sensor 530 into an intraocular pressure measurement based upon the particular parameters of the monitoring system 500, which is then stored in the memory module 176. The electrical current will then be shut off and the same process will be repeated in the contralateral eye. The monitoring system 100 may be programmed to reactivate on a regular interval to repeat intraocular pressure tests (e.g., about every five minutes, about every ten minutes, about every twenty minute, about every sixty minutes, or more). As described above, the data related to the information from the pressure sensor 530 can be transmitted to a computer system, or the like, via a wired or wireless connection. The data can be used for subsequent calculations, for display to the user and/or a physician. The data can also be used as part of a pressure profile.

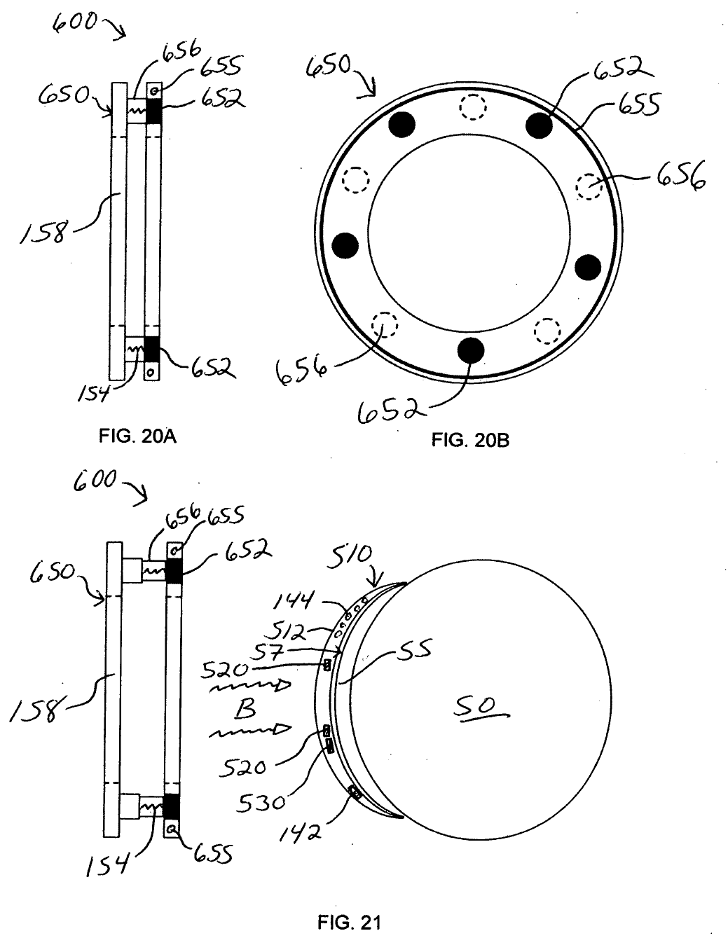

Referring now to FIGS. 20-21, some embodiments of a monitoring system 600 may include a headset device 650 that includes one or more permanent magnets 652 that generate a magnetic field B to impose a force upon the magnet 520 disposed in the contact lens device 510. The headset device 650 can include one or more slide rails 656 that allow the magnets 652 to be moved closer to or farther away from the frame 158. As such, the magnets can be moved closer to the frame 158, as shown in FIG. 20A, moving the magnets farther away from the contact lens device 510 (FIG. 21). This has the effect of decreasing the effect of the magnetic field B on the contact lens device 510, thereby decreasing the force applied from the headset device 650 to the contact lens device 510 via the magnetic field B. When the monitoring system 600 is activated to measure the intraocular pressure of the eye 50, the magnets 652 may be moved, along the rails 656, away from the frame 158 and toward the contact lens device 510 (as shown in FIG. 21) until the force sensor 154 indicates that the predetermined amount of force (e.g., between about 9.8 and 39.4 mN, about 12 mN, about 31.7 mN, or the like) has been reached. As previously described, when the force sensor 154 indicates that the predetermined force has been reached, at least a portion of the cornea 55 is deformed such that the curvature of at least a portion of the cornea 55 substantially matches the curvature of an abutting portion of the contact lens device 510. A pressure measurement, detected by the pressure sensor 530, can then be communicated via the antenna 144 to the corresponding antenna 655 on the headset device 650 for storage in the memory module 176 (refer to FIG. 12). The stored pressure measurement can be used to determine the intraocular pressure of the patient's eye 50. This same process can be repeated in the contralateral eye. The monitoring system 600 may be programmed to reactivate on a regular interval to repeat intraocular pressure tests (e.g., about every five minutes, about every ten minutes, about every twenty minute, about every sixty minutes, or more).

It should be understood from the description herein that, in alternative embodiments, the headset device 650 having the permanent magnets 652 may be implemented for use with the contact lens device 110 or 410 described in connection with FIGS. 1-15. In such embodiments, the magnets 652 of the head set device 650 can be used to provide a magnetic field that acts upon the magnet 120 in contact lens device 110 or 410 to provide the previously described deformation.

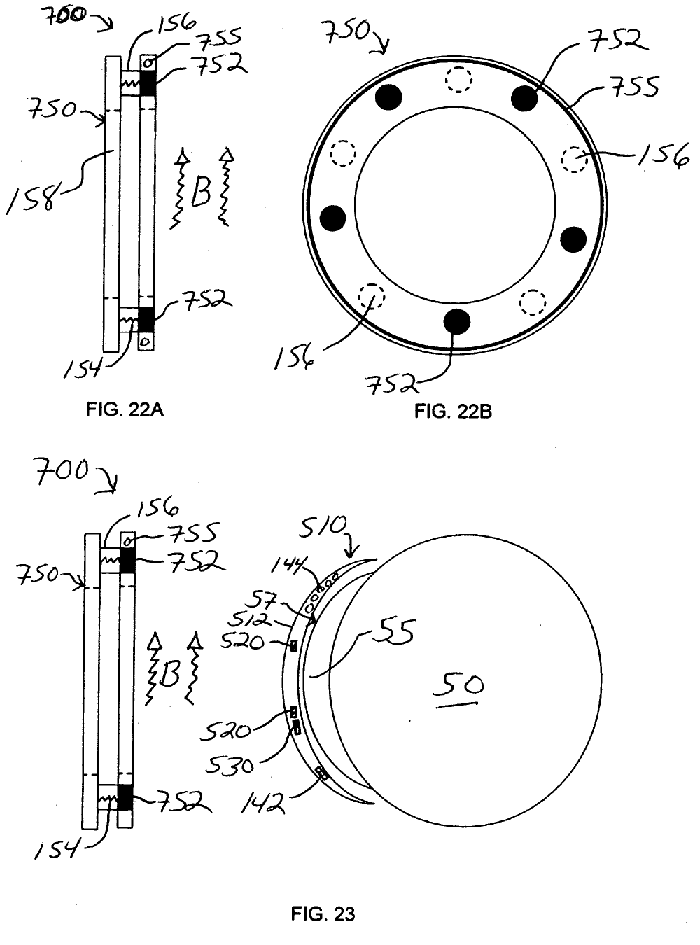

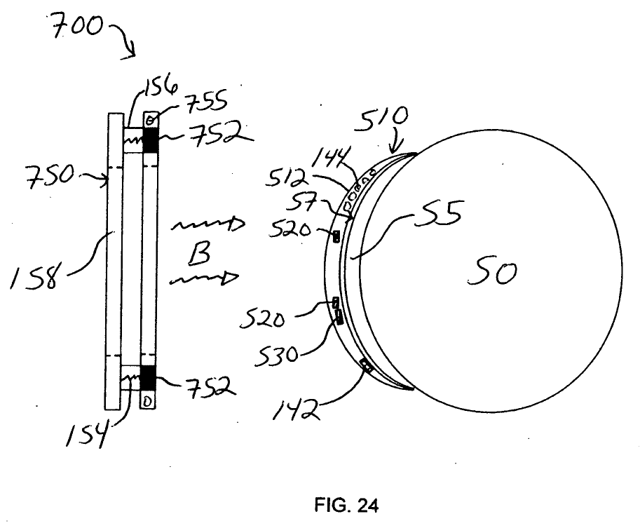

Referring now to FIGS. 22-24, some embodiments of a monitoring system 700 may include a headset device 750 that includes one or more permanent magnets 752 that generate a magnetic field B to impose a force upon the magnet 520 disposed in the contact lens device 510. The magnets 752 can be rotated with respect to the frame of the headset device 750 to alter the direction of polarity for the purpose of adjusting the force applied from the headset device 750 to the contact lens device 510. In a resting state, as shown in FIG. 23, the magnets 752 can be rotated such that the polarity of the magnets 752 is perpendicular to the polarity of the magnet 520 in the contact lens device 510. In this state, no force is being applied from the headset device 750 to the contact lens device 510. When the monitoring system 700 is activated to measure the intraocular pressure of the eye 50, the magnets 752 may be rotated toward the orientation shown in FIG. 24, where the polarity of the contact lens magnet 520 and the magnets 752 are aligned. The magnets can be aligned toward the orientation shown in FIG. 24 until the sensor 154 indicates that the predetermined amount of force (e.g., between about 9.8 and 39.4 mN, about 12 mN, about 31.7 mN, or the like) has been reached. As previously described, when the force sensor 154 indicates that the predetermined force has been reached, the contact lens device 510 is slightly deformed such that the curvature the cornea 55 substantially matches the inner contour of the abutting portion of the contact lens device 510. A pressure measurement can be detected by the pressure sensor 530 and then communicated via the antenna 144 to the corresponding antenna 755 on the headset device 750 for storage in the memory module 176 (refer to FIG. 12). The stored pressure measurement can be used to determine the intraocular pressure of the patient's eye 50. This same process can be repeated in the contralateral eye. The monitoring system 700 may be programmed to reactivate on a regular interval to repeat intraocular pressure tests (e.g., about every five minutes, about every ten minutes, about every twenty minute, about every sixty minutes, or more). Here again, it should be understood from the description herein that, in alternative embodiments, the headset device 750 having the permanent magnets 752 may be implemented for use with the contact lens device 110 or 410 described in connection with FIGS. 1-15. In such embodiments, the magnets 752 of the head set device 750 can be used to provide a magnetic field that acts upon the magnet 120 in contact lens device 110 or 410 to provide the previously described deformation.

In some embodiments, a combination of permanent magnets (such as those described in connection with FIGS. 20-24) can be employed in addition to electromagnets (as described in connection with FIGS. 18-19). In these embodiments, the force applied by the permanent magnets can augment the force applied by the electromagnets, thus advantageously reducing the power consumption and/or weight of the headset.

It should be understood that the system 100 described herein may be employed to determine other ocular factors in addition to the intraocular pressure. For example, the system 100 can be used to determine the aqueous outflow facility of a patient's eye. Aqueous outflow facility is a measure of the ease with which fluid within the anterior chamber of the eye can exit. It may be characterized as the inverse of fluid resistance. Outflow facility can be impaired in glaucoma and the degree of impairment may be related to the severity of the disease. Typically, a method of measuring outflow facility in living patients is with the process of tonography in which an electronic Schiotz tonometer or pneumatonometer is used to apply a steady force directly to the cornea, causing an indentation, and creating an artifactual increase in the intraocular pressure. As the force is maintained, fluid will exit the eye at an increased rate, resulting in a decay of the intraocular pressure towards its normal steady state. This conventional method can be highly user dependent, requiring a significant amount of training and skill, and this method can be difficult for the patient because the constant force must be applied to the patient's eye for 2 to 4 minutes.

The monitoring system 100 described herein can provide a convenient and accurate measure of outflow facility. In some embodiments, the monitoring system 100 can be used to apply a constant indentation in the patient's eye (rather than applying a constant force directly with a Schiotz tonometer). It should be understood that the indentation may be significantly larger than that previously described in connection with the intraocular pressure measurements because the pressure would need to be artifactually increased. In these embodiments, the intraocular pressure can be repeatedly measured (e.g., every 1 second) using a substantially similar process as that described above in connection with FIGS. 1-16. The decay in intraocular pressure under constant indentation could thus be measured and used to determine outflow facility. As previously described, the used of the monitoring system 100 may provide accurate results that are less dependent on the technical skill of the operator. Also, the monitoring system 100 may be capable of collecting the intraocular pressure data used to determine the outflow facility in a shorter period of time (e.g., less than the conventional 2 to 4 minutes). Further, the monitoring system 100 may be capable of determining the outflow facility while the patient is in the nocturnal phase.

A number of embodiments of the invention have been described. Nevertheless, it will be understood that various modifications may be made without departing from the spirit and scope of the invention. Accordingly, other embodiments are within the scope of the following claims.

What is claimed is:

1. An intraocular pressure monitoring system, comprising:
a contact lens device that is removably engageable with an eye, the contact lens device including a sensor device to detect when a deformable portion of the contact lens device is indented by a predetermined amount;
a headset device that applies a force to indent the deformable portion of the contact lens device, the headset device including at least one force sensor coupled to a headset frame; and
a control system to activate the headset device to apply the force on the contact lens device, the control system being in electrical communication with the force sensor to record data from the force sensor when the contact lens device is indented by the predetermined amount.

2. The system of claim 1, wherein the at least one force sensor of the headset device provides data while the contact lens device operates free of a force sensor.

3. The system of claim 1, wherein the contact lens device comprises a magnet in the deformable portion, the deformable portion having no force sensor embedded therein.

4. The system of claim 1, wherein the headset device is activated to apply the force on the contact lens device at a regular interval over an extended period of time.

5. The system of claim 4, wherein the headset device is activated to apply the force on the contact lens device at the regular interval selected from the group consisting of every five minutes, every ten minutes, every twenty minutes, and every sixty minutes.

6. The system of claim 4, wherein the headset device is activated to apply the force on the contact lens device over the extended period of time selected from the group consisting of six hours, twelve hours, and twenty-four hours.

7. The system of claim 1, wherein the control system records data to monitor the intraocular pressure both when the eye is opened and when the eye is closed.

8. The system of claim 7, wherein the contact lens device and the headset device are wearable by a sleeping user so that the control system collects data when the user is in a nocturnal phase.

9. The system of claim 8, wherein the control system collects data from the force sensor of the headset device to generate an intraocular pressure profile recorded over at least a 24-hour period.

10. The system of claim 1, wherein the headset device and contact lens device cooperate to passively measure a force without manual activation by the user.

11. The system of claim 1, wherein the headset device interacts with the contact lens device to provide a nonsurgical system to monitor intraocular pressure.

12. The system of claim 1, wherein the deformable portion of the contact lens device is at least partially displaced in response to a magnetic field generated from the headset device.

13. The system of claim 12, wherein contact lens device includes a magnet attached to the deformable portion.

14. The system of claim 13, wherein the contact lens device further comprises an RFID tag and an antenna line to connect with the RFID tag.

15. The system of claim 14, wherein the sensor device of the contact lens device comprises a break in the antenna line formed when the deformable portion is indented by the predetermined amount.

16. The system of claim 14, wherein the deformable portion is at least partially defined by a groove formed in contact lens material.

17. The system of claim 16, wherein the sensor device of the contact lens device comprises conductive contact elements in communication with the antenna line, the conductive contact elements being separated across the groove when the deformable portion is indented by the predetermined amount.

18. The system of claim 1, wherein the headset device comprises at least one induction coil coupled to the frame.

19. The system of claim 18, wherein the induction coil of the headset device generates a magnetic field that acts upon a magnet of the contact lens device.

20. The system of claim 18, wherein the headset device comprises a pair of induction coils coupled to the frame, the system further comprising a second contact lens device that is removably engageable with a second eye such that the pair of induction coils are positionable proximate to the first and second contact lens devices.

21. The system of claim 1, wherein the headset frame comprises a wearable frame for eyeglasses.

22. The system of claim 21, wherein at least a portion of the control system is housed by the wearable frame.

23. The system of claim 1, wherein at least a portion of the control system is wearable by a user.

24. The system of claim 1, wherein the control system includes a controller circuit to selectively activate the headset device, a rechargeable battery that provides electrical power to the headset device, and a memory module to record data from the force sensor.

25. A contact lens device for use in an intraocular pressure monitoring system, the contact lens device comprising:
a soft contact material that is removably engageable with an eye;
a magnet attached to a deformable portion of the soft contact material;
a RFID device to communicate a code when activated, the RFID device being attached to the soft contact material;
an antenna line to wirelessly communicate the code provided by the RFID device, the antenna line being attached to the soft contact material; and
a switch device to indicate when the deformable portion of the contact lens device is displaced by a predetermined amount.

26. The contact lens device of claim 25, wherein the switch device cuts off at least a portion of the antenna line from the RFID device when the deformable portion is displaced by the predetermined amount.

27. The contact lens device of claim 26, wherein the switch device comprises a break in the antenna line formed when the deformable portion is displaced by the predetermined amount.

28. The contact lens device of claim 26, wherein the deformable portion is at least partially defined by a groove formed in contact lens material.

29. The contact lens device of claim 28, wherein the sensor device of the contact lens device comprises conductive contact elements in communication with the antenna line, the conductive contact elements being separated across the groove when the deformable portion is displaced by the predetermined amount.

30. The contact lens device of claim 25, wherein the deformable portion of the contact lens device is at least partially displaced in response to a magnetic field generated from an external source.

31. The contact lens device of claim 30, wherein the RFID device comprises an RFID tag to output a code to an RFID reader arranged external to the contact lens device.

32. The contact lens device of claim 31, wherein the antenna communicates the code from the RFID tag in response to a magnetic field while the contact lens device operates free of a force sensor.

33. The contact lens device of claim 32, wherein the antenna communicates the code from the RFID tag both when the eye is opened and when the eye is closed.

34. The contact lens device of claim 33, wherein the contact lens device is wearable by a sleeping user so that the antenna communicates the code from the RFID tag when the user is in a nocturnal phase.

35. A headset device for use in an intraocular pressure monitoring system, the headset device comprising:
a frame that is wearable on a head of a user;
at least one induction coil coupled to the frame so that the induction coil is arranged proximate an eye when the frame is worn on the head, the induction coil generating a magnetic field to act upon a magnet external to the headset device; and
a force sensor coupled to the frame to detect a force applied to the induction coil.

36. The headset device of claim 35, further comprising a communication line to transmit data from the force sensor to a control system.

37. The headset device of claim 36, wherein at least a portion of the control system is housed by the wearable frame.

38. The headset device of claim 35, wherein the headset device comprises a pair of induction coils coupled to the frame so that the pair of induction coils are arranged proximate a pair of eyes when the frame is worn on the head.

39. The headset device of claim 35, wherein the wearable frame comprises a frame for eyeglasses.

40. A method for monitoring intraocular pressure, comprising
activating at least one induction coil to generate a magnetic field that applies a force to a contact lens device removably engaged with an eye, the induction coil being coupled to a wearable frame of a headset device so that the induction coil is arranged proximate to the eye;
receiving data from a force sensor arranged on the headset device while the magnetic field is being generated by the induction coil;
detecting a change in a wireless signal from the contact lens device;
recording data from the force sensor when the change in the wireless signal is detected.

41. The method of claim 40, further comprising deactivating the induction coil to shut off the magnetic field when the change in the wireless signal is detected.

42. The method of claim 40, further increasing the intensity of the magnetic field generated by the induction coil until the change in the wireless signal is detected.

43. The method of claim 40, repeating the activating operation, the receiving operation, the detecting operation, and the recording operation at a regular interval over an extended period of time.

44. The method of claim 43, wherein the operations are repeated at the regular interval selected from the group consisting of every five minutes, every ten minutes, every twenty minutes, and every sixty minutes.

45. The method of claim 43, wherein the operations are repeated over the extended period of time selected from the group consisting of six hours, twelve hours, and twenty-four hours.

46. The method of claim 43, further comprising collecting data from the force sensor of the headset device to generate an intraocular pressure profile recorded over at least a 24-hour period.

47. A method for monitoring intraocular pressure, comprising
applying a magnetic field from a headset device to magnet coupled with contact lens device removably engaged with an eye, the headset device comprising wearable frame so that the headset device arranged proximate to the eye;
receiving information indicative of a tear film pressure between the contact lens device and a cornea of the eye, the tear film pressure being detected by a pressure sensor coupled with contact lens device while the magnetic field is being applied to the magnet coupled with contact lens device;
storing data indicative of an intraocular pressure based upon the information indicative of the tear film pressure between the contact lens device and the cornea.

48. The method of claim 43, wherein the operations are repeated over the extended period of time selected from the group consisting of six hours, twelve hours, and twenty-four hours.

49. The method of claim 48, generating an intraocular pressure profile recorded over the extended period of time.
